(19) United States
(12) Patent Application Publication
(10) Pub. No.: US 2026/0315103 A1
(43) Pub. Date: Oct. 15, 2026

(54) ECHOGENIC NEEDLE

(71) Applicant: LICAN DEVELOPMENTS, LTD, Amherstburg (CA)

(72) Inventor: STEVE LIVNEH, NORTHGLENN, CO (US)

(73) Assignee: LICAN DEVELOPMENTS, LTD, Amherstburg (CA)

(21) Appl. No.: 19/333,976

(22) Filed: Sep. 19, 2025

Related U.S. Application Data
(60) Provisional application No. 63/696,610, filed on Sep. 19, 2024.

(51) Int. Cl. A61B 17/34; A61B 90/00

(52) U.S. Cl. CPC A61B 17/3403; A61B 17/3417; A61B 90/39; A61B 2017/3413; A61B 2017/3454; A61B 2090/3925

(57) ABSTRACT

An echogenic needle includes a hollow lumen with a sharp distal end and a connecting hub at a proximal end. The hollow lumen is formed to be grooved on its outer surface with one or more parallel or helical grooved domains, commencing at a short distance behind the sharp tip of the needle. The grooves' geometry is designed to provide effective reflection of ultrasound waves. The reflection enhances the visibility of the echogenic needle on an ultrasound machine's screen, once the ultrasound hand-held probe is aligned in good position on the patient's skin, in the vicinity to the needle that is being inserted into the target tissue. The grooves' geometry achieves effective reflection of the ultrasound waves, resulting in a clear picture on the ultrasound machine's screen, thus providing clear guidance to the user in placing the needle's tip at the desired location within the patient's tissue.

### CROSS-REFERENCE TO RELATED APPLICATION

The instant application claims priority to U.S. Provisional Patent Application Ser. No. 63/696,610, filed Sep. 19, 2024, pending, the entire specification of which is expressly incorporated herein by reference.

### FIELD OF THE INVENTION

The present invention relates generally to medical echogenic devices, and more particularly, to a medical echogenic needle or cannula operable to be inserted into living tissue under ultrasound guidance, wherein the echogenic properties of the medical device effectively enhance the accurate placement of the medical device's distal tip, when viewed with an ultrasound machine with a screen or monitor.

### BACKGROUND OF THE INVENTION

Echogenic needles appeared as early as the late 1960s when ultrasound usage became a recognized medical practice and was applied in procedures such as guided renal biopsies. The sonographic visualization provided by the usage of ultrasound transducers led to the rapid development of the interventional ultrasound medical field. Naturally, enhanced visualization on the ultrasound machine's monitor was highly desired. This led to the continuous development of echogenic needles, cannulas and cables, enhancing the visibility of the inserted devices when guided by and viewed on ultrasound machines.

However, conventional echogenic needles suffer from several deficiencies, including, but not limited to, difficulty with respect to the accurate placement of the distal tip of the echogenic needle when placed into the patient's tissue.

Accordingly, there exists a need for new and improved medical echogenic devices, especially medical echogenic needles or cannulas that overcome the aforementioned deficiencies.

### SUMMARY OF THE INVENTION

The present invention describes an echogenic device, such as but not limited to a needle, which, in essence, is similar to standard needles of the same gauge and length but with added echogenic formations on the needle's lumen outer surface. These formations are designed to reflect ultrasound waves back to the ultrasound probe. The reflection is sensed by the ultrasound machine and is shown on the screen as a distinct line. This feature assists users in directing the echogenic device, such as but not limited to a needle, at greater accuracy to the desired point of injection, biopsy and/or delivery of an internal working element.

The present invention focuses on the specific type and geometry of the echogenic formation on the described echogenic needle. Identical formations may be applied on other medical devices that also require enhanced echogenicity.

The present invention also describes an echogenic needle. An echogenic needle is similar in most details to standard needles as described herein.

By way of a non-limiting example, the needle includes a metallic hollow shaft, referred to as a lumen, and may be commonly made of stainless-steel alloy and/or the like.

The lumen distal end is preferably sharpened as per defined standards that apply to medical needles.

The sharp distal end, or tip, allows for easy penetration into the tissue with reduced injury and pain to the patient.

The proximal end of the lumen is preferably attached to a hub, made either of plastic, metal and/or some other suitable material. The hub allows for quick attachment and/or detachment to standard syringes while sealing the connection area against leakage of a fluid or air.

The needles are typically classified by the gauge of the lumen and the effective length of the lumen. The effective length is generally measured from the tip to the point where the lumen enters the proximal hub.

The echogenic needle that is described herein is preferably comprised of all the elements, as set forth above, with the added echogenic formations placed in close proximity to the sharp tip of the lumen. The formations are preferably placed as one or more domains or zones, typically about 10 mm long. Shorter needles may come with one domain, typically about a 10 mm long formed domain on the lumen. The formed domain preferably begins at about 5 mm behind the needle's tip. Longer length needles may come with two or more such formed domains, preferably spaced at about 5 mm apart.

The echogenic formations, and their placement, are the primary subject of the present invention.

The echogenic formations of the present invention are preferably formed as parallel or helical grooves on the outside surface of the lumen. The grooves are preferably formed in a V-shape, with an included angle α in the range of 60°-150°. The preferred angles for enhanced echogenicity are in the range of 91°-135°, as they provide relatively good echogenic reflection back at the ultrasound probe from common angles used by interventional radiologists, clinicians and technicians. In accordance with a still more preferred embodiment, the angles for enhanced echogenicity are in the range of 91°-120°.

The advantage of the helical grooves is in being less abrasive to the tissue during penetration into the patient's tissue. The advantage of the parallel grooves is in better sonographic signature.

Echogenic needles according to the present invention may be offered in a variety of gauges and lengths, such as but not limited to those in the range of 20-25 gauge and lengths between 1-6 inches (i.e., 25.4-152.4 mm).

In accordance with the general teachings of the present invention, there is provided a needle with one or more of the following features:
(1) The needle is comprised of a stainless-steel lumen;
(2) The lumen has a sharp distal end;
(3) The lumen proximal end is permanently attached to a hub, made either of plastic, metal and/or another suitable material; and
(4) The external circumference surface of the lumen is formed into echogenic grooves, for example, either parallel or in a helix orientation. The grooves are preferably formed in sections, such as but not limited to 8-12 mm long. In accordance with a more preferred embodiment, the grooves are formed in sections of 10 mm long. The distal end of the first section commences preferably at 5 mm behind the needle's sharp tip. One or more grooved domains may be formed on the lumen, with a preferred gap of 4-6 mm between each section. In accordance with a more preferred embodiment, one or more grooved domains may be formed on the lumen with a gap of 5 mm between each section.

The echogenic formations are preferably formed by mechanical forming means, with emphasis of the grooves' included angle and surface finish. As previously noted, a preferred groove configuration includes an angle in the range of 91°-135°. An opening angle in this range is thought to provide a relatively long groove wall, so as to act as a reflective surface.

By way of a non-limiting example, one preferred groove depth is in the range of 0.015-0.08 mm. The groove's bottom should preferably have a minimal radius or flat portion, so as to allow for maximal width of the reflective walls.

The echogenic grooves are preferably arranged in domains of multiple grooves. The domain length is preferably about 10 mm with a preferred gap between domains of 5 mm. Adding domains to the longer needles at or above 50 mm, or about 2″, adds clarity when viewed on the ultrasound machine monitor. The number of grooves within a domain is preferably a function of the groove's opening angle, depth and/or pitch.

The grooves density or pitch is generally defined as the linear distance between two adjacent grooves centers. The pitch preferably includes the total groove's width and the length of an unformed region between adjacent grooves. The groove's pitch is preferably in the range of 0.5-1.5 mm. In accordance with a more preferred embodiment, the groove's pitch is in the range of 0.5-0.8 mm.

The quality of the image of the echogenic needle as shown on the ultrasound machine is greatly dependent of the groove's walls surface finish. The smoother the walls are, the stronger the reflection is and, hence, better visualization on the ultrasound machine's monitor.

By way of a non-limiting example the echogenic grooves are preferably comprised of parallel grooves with symmetrical walls and include an angle α preferably ranging between 60°-150°, and still more preferably in the range of 91-135°. The arms of the V-shaped groove are preferably connected to each other with a small radius or flat or arced narrow unformed gap. The grooves are preferably formed on the lumen in domains, i.e., groups. A preferred domain length is about 10 mm.

The grooves may be formed with a forward tilt referred herein as β, for example, towards the tip. The tilt angle β may preferably be in the range of 3°-40°. Still more preferably, the tilt angle β may preferably be in the range of 25°-40°. It is by definition the angular forward-tilt of the included groove angle α.

The advantage in tilting the groove forward is found in shallow angle injections, where the forward tilt causes the distal groove's arm to appear closer to 45-60°for more effective reflection back to the ultrasound probe.

The grooves may be formed in a helix, with the same range of pitch densities, i.e., in the range of 0.5-1.2 mm. The helix angle is dictated by the selected groove pitch. As an example, for grooves pitches of 0.5 mm on a 23-gauge lumen with an OD of 0.6414 mm, the helix angle is at:

Arctangent(P/π×OD)=arctangent(0.5/π×0.6414)=13.93°.

As with the parallel grooves, the helical grooves may be formed with a tilt angle β towards the tip. As noted above, the tilt angle may be in the range of 3°-40°. Also as noted above, still more preferably the tilt angle β may preferably be in the range of 25°-40°.

Another important aspect of the present invention is the inclusion of a formed depth-marker at a given distance proximally to the rearmost echogenic formation domain. Currently, there are laser-etched markings of the lumens of certain echogenic needles. These cannot be seen echogenically when embedded in the patient's tissue. However, the formed depth-marker of the present invention is easily visible both outside the patient's tissue and by the ultrasound machine when within the patient'stissue.

The present invention also provides advanced echogenic groove forms. These detailed forms allow advanced forming techniques while slowing good forming tools' life. At the same time, these forms provide good echogenicity while greatly reducing abrasion to the patients' skin and internal tissues. The feature that enhances reduced-pain during penetration is in the shoulder-radius of the V-shaped groove walls' ends, i.e., the radius that connects the groove's wall to the lumen's external surface.

Two preferred groove forms are shown:
(1) A flat-bottomed, rounded-shoulder groove type used in both in parallel and helical echogenic domains. The flat bottom allows certain types of mechanical forming while providing tool longevity. The rounded groove's shoulders provide significant reduction in tissue abrasion during penetration and, hence, much reduced pain to the patient; and
(2) A round-bottomed, rounded-shoulder groove type used in both parallel and/or helical echogenic domains. The round bottom allows certain types of mechanical forming while providing tool longevity. The rounded groove's shoulders provide significant reduction in tissue abrasion during penetration and, hence, much reduced pain to the patient.

In accordance with a first embodiment of the present invention, an echogenic needle is provided, comprising:
an elongated, cylindrical member having a proximal end and a distal end and an area defining a throughbore formed between the proximal end and the distal end;
wherein the distal end includes an angled portion operable to penetrate human tissue;
a first plurality of parallel V-shaped groove portions formed on an outer surface of the member;
wherein the V-shaped groove portions include an angle in a range of 60°-150°;
wherein a bottom surface of the V-shaped groove portions is substantially flat or has a radius equal to or less than 0.05 mm.

In an aspect of the first embodiment, the V-shaped groove portions include an angle in a range of 91°-136°.

In an aspect of the first embodiment, the first plurality of parallel V-shaped groove portions is formed in a first domain in a range of 8-12 mm long.

In an aspect of the first embodiment, the first domain of the first plurality of parallel V-shaped groove portions commences at 5 mm or less from the distal end of the member.

In an aspect of the first embodiment, a second plurality of parallel V-shaped groove portions is formed on the outer surface of the member.

In an aspect of the first embodiment, the second plurality of parallel V-shaped groove portions include an angle in a range of 60°-150°.

In an aspect of the first embodiment, a bottom surface of the second plurality of parallel V-shaped groove portions is substantially flat or has a radius equal to or less than 0.05 mm.

In an aspect of the first embodiment, the second plurality of parallel V-shaped groove portions include an angle in a range of 91°-136°.

In an aspect of the first embodiment, the second plurality of parallel V-shaped groove portions is formed in a second domain in a range of 8-12 mm long.

In an aspect of the first embodiment, the first domain is spaced 4-6 mm from the second domain.

In an aspect of the first embodiment, the V-shaped groove portions have a depth in a range of 0.015-0.08 mm and a pitch in a range of 0.5-1.2 mm.

In an aspect of the first embodiment, the V-shaped groove portions are tilted towards the distal end of the member with a tilt angle in a range of 3°-40°.

In an aspect of the first embodiment, the V-shaped groove portions include an area defining a shoulder surface having a radius in a range of 0.02-0.06 mm.

In an aspect of the first embodiment, a depth marker is provided, wherein the depth marker comprises at least one groove portion formed on the outer surface of the member, wherein the depth marker is spaced proximally apart from the first plurality of parallel V-shaped groove portions.

In accordance with a second embodiment of the present invention, an echogenic needle is provided, comprising:
an elongated, cylindrical member having a proximal end and a distal end and an area defining a throughbore formed between the proximal end and the distal end;
wherein the distal end includes an angled portion operable to penetrate human tissue;
a first plurality of helical V-shaped groove portions formed on an outer surface of the member;
wherein the V-shaped groove portions include an angle in a range of 60°-150°;
wherein a bottom surface of the V-shaped groove portions is substantially flat or has a radius equal to or less than 0.05 mm.

In an aspect of the second embodiment, the V-shaped groove portions include an angle in a range of 91°-136°.

In an aspect of the second embodiment, the first plurality of helical V-shaped groove portions is formed in a first domain in a range of 8-12 mm long.

In an aspect of the second embodiment, the first domain of the first plurality of helical V-shaped groove portions commences at 5 mm or less from the distal end of the member.

In an aspect of the second embodiment, a second plurality of helical V-shaped groove portions is formed on the outer surface of the member.

In an aspect of the second embodiment, the V-shaped groove portions include an angle in a range of 60°-150°.

In an aspect of the second embodiment, a bottom surface of the V-shaped groove portions is substantially flat or has a radius equal to or less than 0.05 mm.

In an aspect of the second embodiment, the V-shaped groove portions include an angle in a range of 91°-136°.

In an aspect of the second embodiment, the second plurality of helical V-shaped groove portions is formed in a second domain in a range of 8-12 mm long.

In an aspect of the second embodiment, the first domain is spaced 4-6 mm from the second domain.

In an aspect of the second embodiment, the second plurality of helical V-shaped groove portions have a depth in a range of 0.015-0.08 mm and a pitch in a range of 0.5-1.2 mm.

In an aspect of the second embodiment, the second plurality of helical V-shaped groove portions are tilted towards the distal end of the member with a tilt angle in a range of 3°-40°.

In an aspect of the second embodiment, the second plurality of helical V-shaped groove portions include an area defining a shoulder surface having a radius in a range of 0.02-0.06 mm.

In an aspect of the second embodiment, a depth marker is provided, wherein the depth marker comprises at least one groove portion formed on the outer surface of the member, wherein the depth marker is spaced proximally apart from the first plurality of helical V-shaped groove portions.

Further areas of applicability of the present invention will become apparent from the detailed description provided hereinafter. It should be understood that the detailed description and specific examples, while indicating the preferred embodiment of the invention, are intended for purposed of illustration only and are not intended to limit the scope of the invention.

### BRIEF DESCRIPTION OF THE DRAWINGS

FIG. 1 illustrates a schematic view of an echogenic needle system in accordance with the general teachings of the present invention, including an echogenic needle, an ultrasound machine's probe, and a monitor operably associated with the ultrasound machine's probe.

FIG. 1A illustrates a schematic view of an echogenic needle in accordance with the general teachings of the present invention shown inserted into a patient's tissue.

FIG. 2A illustrates a schematic view of an echogenic needle in accordance with a first embodiment of the present invention, with parallel grooves, in domains, and a formed depth marker.

FIG. 2B illustrates an enlarged detail view A of a distal portion of the echogenic needle shown in FIG. 2A, with parallel grooves formed thereon.

FIG. 2C illustrates an enlarged detail view B of the parallel grooves shown in FIG. 2B.

FIG. 3A illustrates a schematic view of an echogenic needle in accordance with a second embodiment of the present invention, with helical grooves, in domains, and a formed depth marker.

FIG. 3B illustrates an enlarged detail view C of a distal portion of the echogenic needle shown in FIG. 3A, with helical grooves formed thereon.

FIG. 3C illustrates an enlarged detail view D of the helical grooves shown in FIG. 3B.

FIG. 4A illustrates a schematic view of a domain of the parallel grooves of the echogenic needle shown in FIG. 2A, formed with a forward tilt angle.

FIG. 4B illustrates an enlarged detail view E of the forward tilted parallel grooves shown in FIG. 4A, with a tilt angle β.

FIG. 4C illustrates a schematic view of a domain of the helical grooves of the echogenic needle shown in FIG. 3A, formed with a forward tilt angle β.

FIG. 4D illustrates an enlarged detail view F of the forward tilted helical grooves shown in FIG. 4C.

FIG. 5A illustrates a schematic view of a flat-bottom parallel-groove domain on an echogenic needle shaft in accordance with a third embodiment of the present invention.

FIG. 5B illustrates an enlarged detail view G of the flat-bottom parallel-groove domain on the echogenic needle shaft shown in FIG. 5A.

FIG. 5C illustrates a schematic view of a flat-bottom helical-groove with rounded shoulders domain on an echogenic needle shaft in accordance with a fourth embodiment of the present invention.

FIG. 5D illustrates an enlarged detail view H of the flat-bottom helical-groove with rounded shoulders domain on the echogenic needle shaft shown in FIG. 5C.

FIG. 6A illustrates a schematic view of a round-bottom parallel-groove domain on an echogenic needle shaft in accordance with a fifth embodiment of the present invention.

FIG. 6B illustrates an enlarged detail view I of the round-bottom parallel-groove domain on the echogenic needle shaft shown in FIG. 6A.

FIG. 6C illustrates a schematic view of a round-bottom parallel-groove domain with rounded shoulders on an echogenic needle shaft in accordance with a sixth embodiment of the present invention.

FIG. 6D illustrates an enlarged detail view J of the round-bottom helical-groove with rounded shoulders domain on the echogenic needle shaft shown in FIG. 6C.

### DETAILED DESCRIPTION OF THE INVENTION

The following description of the preferred embodiment(s) is merely exemplary in nature and is in no way intended to limit the invention, or uses.

### Legend

2—Echogenic needle system
4—Cable
10—Echogenic needle with parallel grooves
12—Proximal portion of echogenic needle
14—Distal portion of echogenic needle
16—Throughbore
20—Echogenic needle with helical grooves
30—Echogenic parallel groove
40—Echogenic helical groove
50—Patient tissue
60—Needle's sharp tip
70—Needle's lumen
80—Hub portion
90—Groove's wall
100—Parallel grooves formed domain
110—Helical groove formed domain
120—Echogenic depth marker
130—Gap between formed sections
140—Ultrasound probe
150—Ultrasound machine's monitor
160—Ultrasound outgoing waves
170—Ultrasound reflected/echoed waves
180—Ultrasound image
190—Echogenic needle image
200—Groove's included angle α
210—Groove's helix angle μ
220—Flat-bottom surface of parallel-groove having a connecting radius
230A, 230B—Parallel flat groove's bottom having a radius
240A, 240B—Parallel grooves shoulder having a radius
250—Round bottom surface of groove bottom having a radius
260A, 260B—Round bottom groove's shoulder having a radius
270—Tilt angle β

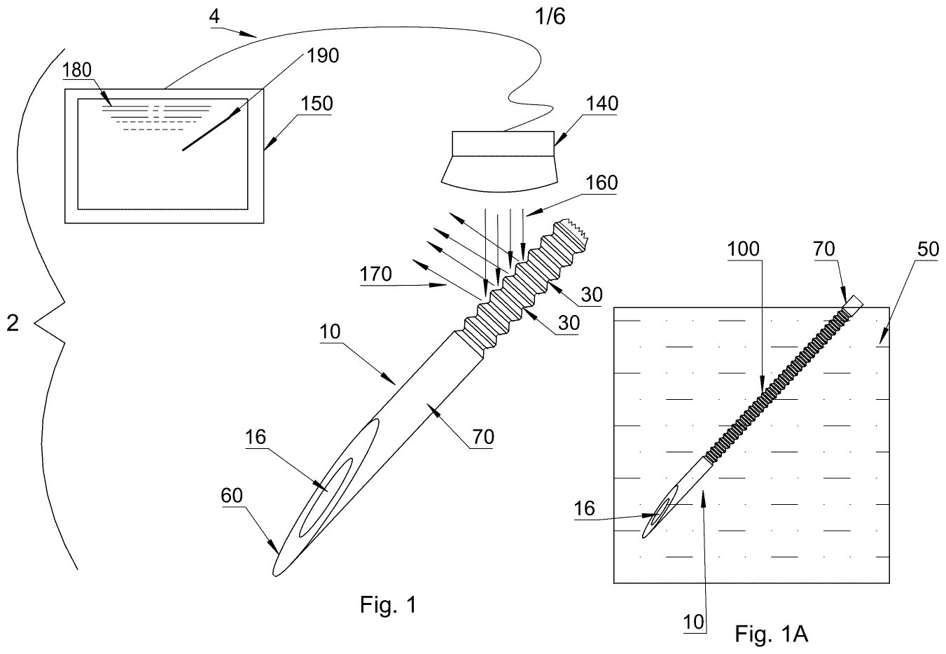

Referring specifically to FIG. 1, there is shown a schematic view of an echogenic needle system 2 in accordance with the general teachings of the present invention, including an echogenic needle 10, an ultrasound machine's probe 140, and a monitor 150 operably associated (e.g., via a cable 4 and/or the like) with the ultrasound machine's probe 140. It should be noted that only a distal portion of the echogenic needle 10 is shown in the view for ease of illustration.

By way of a non-limiting example and referring specifically to FIG. 1A, the echogenic needle 10 is intended to be inserted into a patient's tissue 50 while being scanned by the ultrasound probe 140 that is operably associated with the monitor 150.

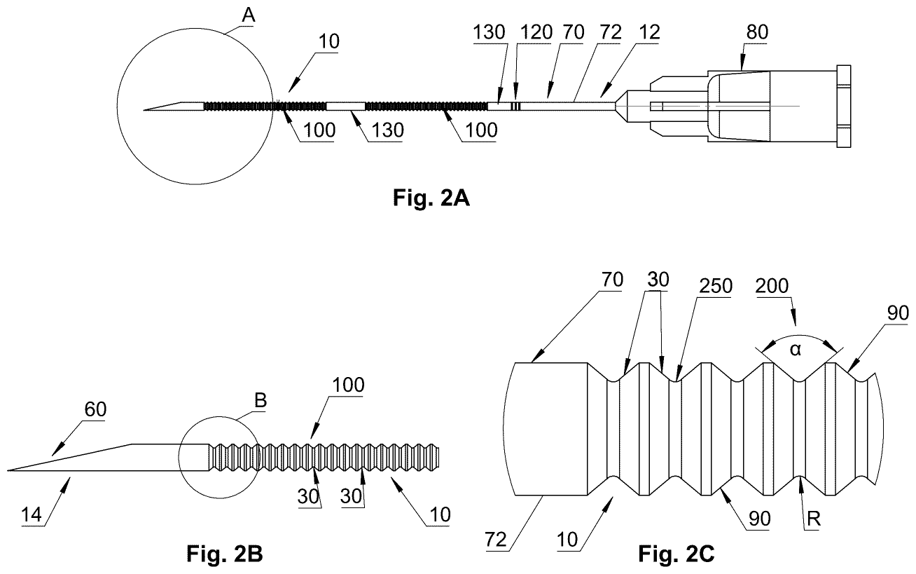

Referring to FIG. 1 and FIGS. 2A-2C, the echogenic needle 10 preferably includes a proximal portion 12, a distal portion 14, and an area defining a throughbore 16 formed therebetween, such that the proximal portion 12 and the distal portion are in fluid communication with one another. The distal portion 14 is preferably provided with a sharp point 60 that is preferably operable to penetrate human tissue. The echogenic needle 10 is preferably comprised of a lumen 70, which is preferably an elongated, cylindrical hollow tube that is permanently attached to a proximal hub portion 80. The external surface 72 of the lumen 70 is preferably formed to have echogenic parallel grooves 30, preferably arranged in groups or domains 100. By the term “parallel,” as that term is used herein, it is meant that the grooves are oriented perpendicular and/or orthogonal (or at least substantially perpendicular and/or orthogonal) to a longitudinal axis extending along the length of the lumen 70,

Although a certain number of echogenic parallel grooves 30 are depicted in this view for purposes of illustration, it should be appreciated that either less than or more than this number of grooves may be used in the practice of the present invention. Although a certain number of domains 100 are depicted in this view for purposes of illustration, it should be appreciated that either less than or more than this number of domains 100 may be used in the practice of the present invention.

In practice, when the echogenic needle 10 is inserted into a patient's tissue 50, the probe 140 preferably emits ultrasound waves 160 (shown here as visible for purposes of clarity). The outgoing waves 160 are then preferably reflected (or echoed) as waves 170 (shown here as visible for purposes of clarity) by the parallel grooves 30. The echoed waves 170 preferably hit the ultrasound probe's receiving unit (not shown) that then sends the processed reflection to the monitor 150 for viewing by the clinician.

The resulting image 180 preferably shows the patient's tissue and structures under the patient skin, together with a clear white or colored line 190. Line 190 preferably shows the relative location of the distal portion 14 of the echogenic needle 10 to the various inner structures of the patient's tissue 50. By way of a non-limiting example, in a case where the echogenic needle 10 is having two formed echogenic domains, being embedded in the patient's tissue, then two lines will be displayed on the monitor 150. This image, as visualized by the clinician giving an injection, for example, aids greatly in directing the echogenic needle 10 to a desired point of delivery of the injection.

Referring specifically to FIG. 2A, there is shown a fully assembled echogenic needle 10 with echogenic parallel grooves 30 formed on lumen 70. Lumen 70 is preferably permanently connected to a hub portion 80. The echogenic needles of the present invention, except for the echogenic formations, are preferably made in the current style of medical subcutaneous needles in materials and form. By way of a non-limiting example, the lumen 70 is preferably made of hypodermic stainless-steel tubing, such as, but not limited to, 304 or 316 alloy. The hub portion 80 is preferably made either of thermoplastic resin or metal.

The echogenic needle 10 may be provided with one or more domains 100 of parallel grooves 30. The first such domain 100 may commence at a preferred distance in the range of 4-6 mm from the sharp point of tip 60. Still more preferably, this distance is 5 mm from the sharp point of tip 60.

A formed echogenic depth marker 120 may preferably be formed proximally to the last (i.e., rear) formed domain 100. This is, in essence, a formed short section of echogenic parallel grooves, wherein its length may preferably be in the range of 3-5 mm. Providing a formed depth marker 120 is advantageous as it allows viewing the marker by ultrasound scanner in the case where the marker gets imbedded in the patient's tissue. The depth marker 120 can be seen by the naked eye as long as it is outside the patient's skin. The distance of the depth marker 120 is set with consideration to the effective needle length, which is equal to the length of the exposed lumen 70 ahead of the hub portion 80.

Although a certain number of echogenic parallel grooves are depicted as forming the echogenic depth marker 120 in this view for purposes of illustration, it should be appreciated that either less than or more than this number of echogenic parallel grooves may be used in the practice of the present invention.

As a non-limiting example, on the lumen of a 23-gauge needle with effective length of 40 mm or approximately 1.5″, the marker center will preferably be at 30 mm or a proximally 1.25″ proximal to the sharp tip 60. This is a very helpful aid to the clinician in assessing the sharp tip 60 location during the insertion of the echogenic needle 10 prior to injecting the patient.

Referring specifically to FIGS. 2A-2C, there is shown the preferred details of the first embodiment's parallel grooves geometry.

By way of a non-limiting example, the preferred groove shape is generally V-shaped, with a preferably included angle α between 60° to 150°, with more preferred range of 91° to 136°. The tip of the V-shaped groove, i.e., the bottom surface of the groove, is shaped preferably as a radius equal to or less than 0.05 mm. Limiting the radius is thought to be essential to leave a maximal reflective surface of the walls 90 for better echogenicity.

The one or more grooves' domains 100 are shown as having a preferable length of 10 mm+/−1 mm.

The domains 100 are preferably separated by an unformed (i.e., smooth) gap 130 having a length of 4-6 mm.

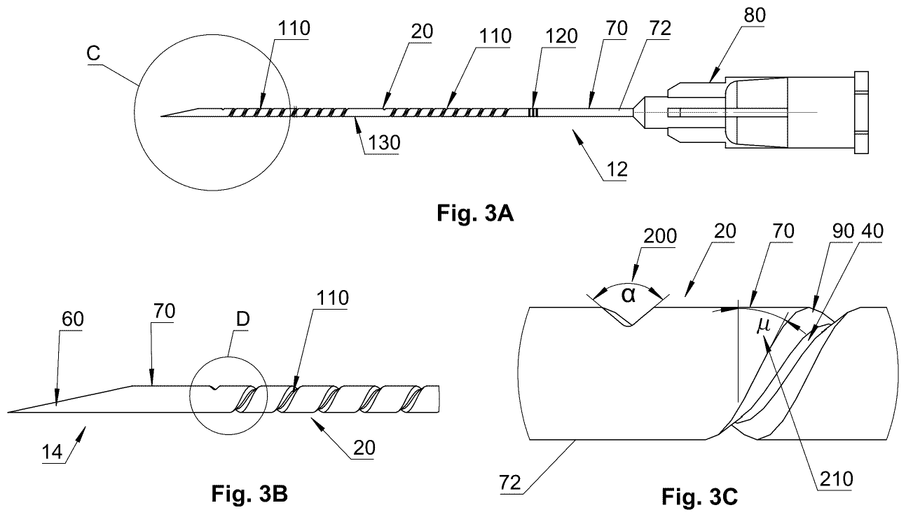

Referring specifically to FIGS. 3A-3C, there is shown a second embodiment of a fully assembled echogenic needle 20 (very similar to the previously described first embodiment) but preferably having echogenic helical grooves 40 (as opposed to echogenic parallel grooves 30) formed on an external surface 72 of lumen 70, and with helix angle μ (also shown as 210) being arranged in domains 110 on lumen 70. Lumen 70, as with the previously described first embodiment, is preferably permanently connected to hub portion 80.

Although a certain number of echogenic helical grooves 40 are depicted in this view for purposes of illustration, it should be appreciated that either less than or more than this number of grooves may be used in the practice of the present invention. Although a certain number of domains 110 are depicted in this view for purposes of illustration, it should be appreciated that either less than or more than this number of domains 110 may be used in the practice of the present invention.

It should also be appreciated that the echogenic depth marker 120 (as previously described) may be provided with echogenic needle 20 as well. Although a certain number of echogenic parallel grooves are depicted as forming the echogenic depth marker 120 in this view for purposes of illustration, it should be appreciated that either less than or more than this number of echogenic parallel grooves may be used in the practice of the present invention.

The echogenic needles are mostly identical to current style subcutaneous needles in materials and form. The lumen is preferably made of hypodermic stainless-steel tubing, like 304 or 316 alloys. The hub portion 80 is preferable made either of thermoplastic resin or metal.

Again, the echogenic needles of the present invention, except for the echogenic formations, are preferably made in the current style of medical subcutaneous needles in materials and form. By way of a non-limiting example, the lumen 70 of echogenic needle 20 is preferably made of hypodermic stainless-steel tubing, such as, but not limited to, 304 or 316 alloy. Again, as with the previously described first embodiment, the hub portion 80 is preferably made either of thermoplastic resin or metal.

Referring specifically, to FIGS. 3B-3C, there is shown the preferred details of the helical grooves' 40 geometry. The preferred groove shape is generally V-shaped, which preferably includes angle α preferably between 60° to 150°, with a more preferred range of 91° to 136°, and a still more preferred range of 91°-120°. The tip of the V-shaped groove, or the bottom of the groove is a radius preferably equal to or less than 0.05 mm. Limiting the radius is thought to be essential to leave a maximal reflective surface of the walls 90 for better echogenicity.

The helical grooves are preferably formed in a helical pattern on the lumen 70. The helix angle μ (also shown as 210) is preferably dictated by the grooves pitch P and the outside diameter (OD) of the lumen 70 (i.e., gauge).

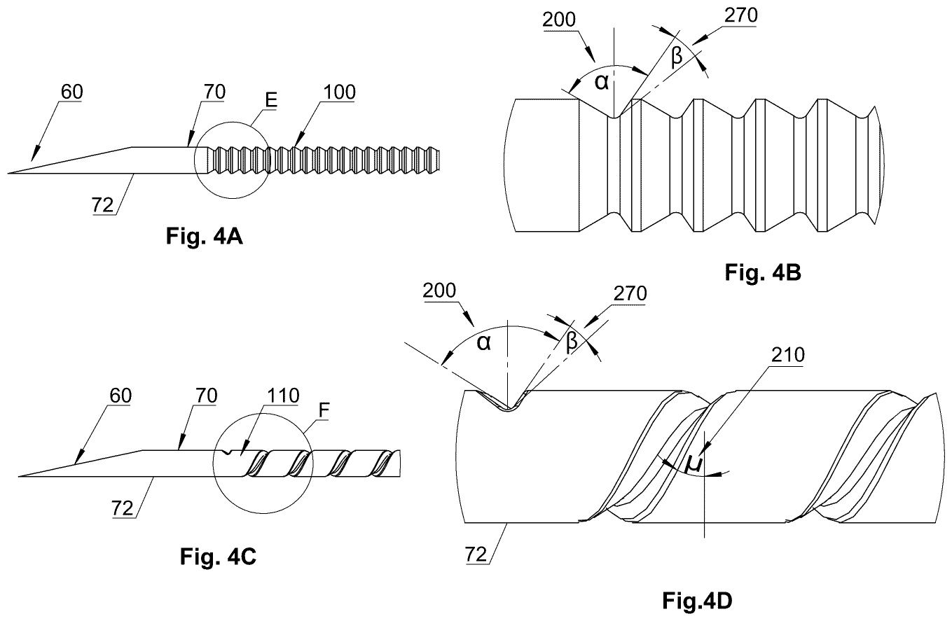

Referring specifically to FIGS. 4A-4B, there is shown an echogenic needle similar to echogenic needle 10 with parallel grooves having a V-shape, wherein an included angle α of the groove is preferably formed with a forward (i.e., towards the tip) tilt angle β (also shown as 270). This feature is thought to be advantageous for applications where the needle is being inserted into the patient's tissue 50 at a relatively shallow angle. The forward tilt preferably aids in better upward echoing of the ultrasound waves towards the scanning probe and eventually enhancing the visualization of the needle on the monitor 150. The forward tilt angle β is preferably in the range of 30-40° that may be optimized for specific applications.

Referring specifically to FIGS. 4C-4D, there is shown an echogenic needle similar to echogenic needle 20 with helical grooves having a V-shape, wherein an included angle α of the groove was formed with a forward (i.e., towards the tip) tilt angle β (also shown as 270). This feature is thought to be advantageous for applications where the needle is being inserted into the tissue in a relatively shallow angle. The forward tilt preferably aids in better upward echoing of the ultrasound waves towards the scanning probe and eventually enhancing the visualization of the needle on the monitor 150.

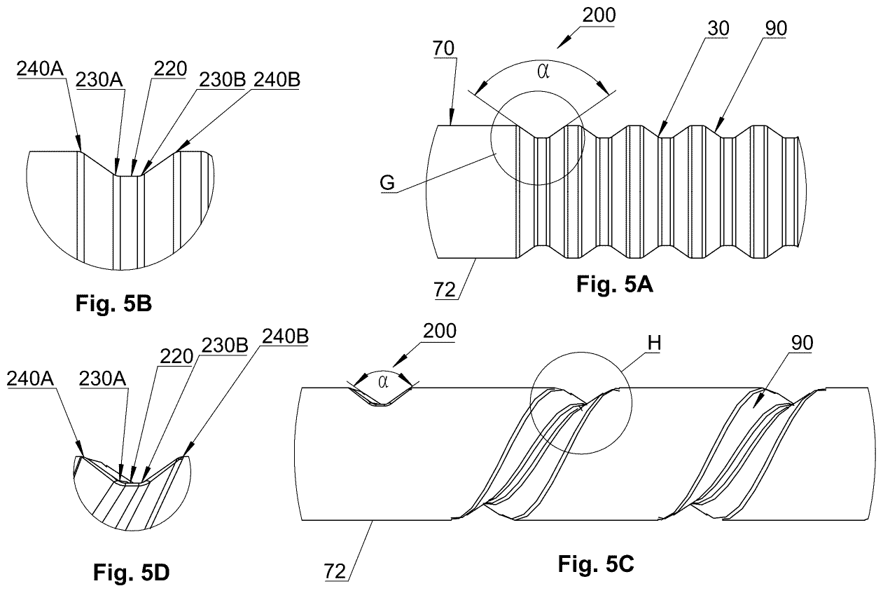

Referring specifically to FIGS. 5A-5B, and in accordance with a third embodiment, there is shown a flat-bottom echogenic parallel groove 30 where the flat bottom surface 220 is preferably in the range of 0.2-0.6 mm long. The flat bottom surface 220 may preferably connect to the groove walls 90 with a small radius 230A, 230B, respectively, preferably in the range of 0.01-0.03 mm. The shoulders 240A, 240B, respectively, of the groove arms preferably connect to the lumen surface 72 with a radius preferably in the range of 0.02-0.06 mm, for example, depending on the groove's depth.

Referring specifically to FIGS. 5C and 5D, and in accordance with a fourth embodiment, there is shown a flat-bottom helical echogenic groove 40 where the flat bottom surface 220 is preferably in the range of 0.02-0.06 mm long. The flat bottom surface 220 may preferably connect to the groove walls 90 with a small radius 230A, 230B, respectively, preferably in the range of 0.01-0.03 mm. The shoulders 240A, 240B, respectively, of the groove arms preferably connect to the lumen surface 72 with a radius preferably in the range of 0.02-0.06 mm, for example, depending on the groove's depth.

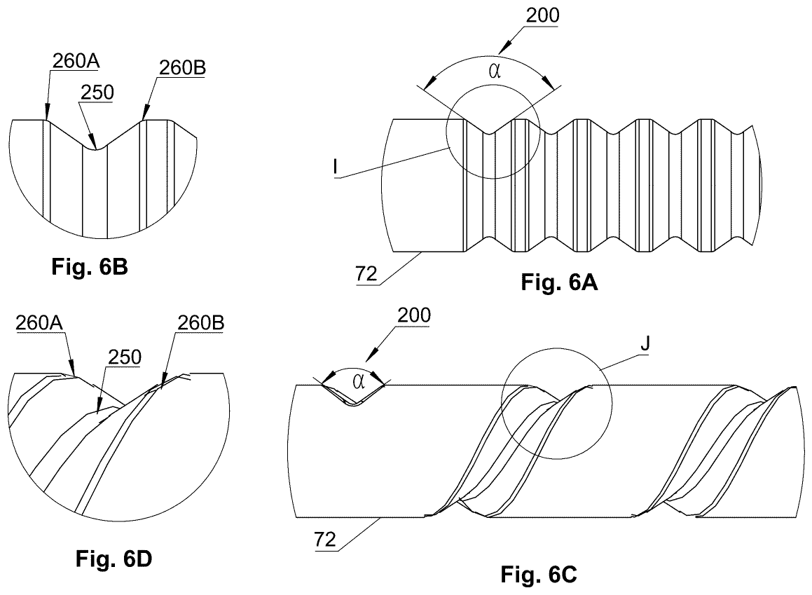

Referring specifically to FIG. 6A and FIG. 6B, and in accordance with a fifth embodiment, there is shown a round-bottom echogenic parallel groove 30 where the round bottom surface 250 is preferably formed as a radius, preferably in the range of 0.03-0.08. The shoulders 260A, 260B, respectively, of the groove arms preferably connect to the lumen surface 72 with a radius preferably in the range of 0.02-0.06 mm, for example, depending on the helical groove's depth.

Referring specifically to FIG. 6C and FIG. 6D, and in accordance with a sixth embodiment, there is shown a round-bottom helical echogenic groove 40 where the round bottom surface 250 is preferably formed as a radius, preferably in the range of 0.03-0.08 mm. The shoulders 260A, 260B, respectively, of the groove arms preferably connect to the lumen surface 72 with a radius preferably in the range of 0.02-0.06 mm, for example, depending on the helical groove's depth.

While the invention has been described with reference to an exemplary embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An echogenic needle, comprising:
an elongated, cylindrical member having a proximal end and a distal end and an area defining a throughbore formed between the proximal end and the distal end;
wherein the distal end includes an angled portion operable to penetrate human tissue;
a first plurality of parallel V-shaped groove portions formed on an outer surface of the member;
wherein the V-shaped groove portions include an angle in a range of 60°-150°;
wherein a bottom surface of the V-shaped groove portions is substantially flat or has a radius equal to or less than 0.05 mm.

2. The invention according to claim 1, wherein the V-shaped groove portions include an angle in a range of 91°-136°.

3. The invention according to claim 1, wherein the first plurality of parallel V-shaped groove portions is formed in a first domain in a range of 8-12 mm long.

4. The invention according to claim 3, the first domain of the first plurality of parallel V-shaped groove portions commences at 5 mm or less from the distal end of the member.

5. The invention according to claim 4, further comprising a second plurality of parallel V-shaped groove portions formed on the outer surface of the member.

6. The invention according to claim 5, wherein the V-shaped groove portions include an angle in a range of 60°-150°.

7. The invention according to claim 6, wherein a bottom surface of the V-shaped groove portions is substantially flat or has a radius equal to or less than 0.05 mm.

8. The invention according to claim 6, wherein the V-shaped groove portions include an angle in a range of 91°-136°.

9. The invention according to claim 5, wherein the second plurality of parallel V-shaped groove portions is formed in a second domain in a range of 8-12 mm long.

10. The invention according to claim 9, wherein the first domain is spaced 4-6 mm from the second domain.

11. The invention according to claim 5, wherein the V-shaped groove portions have a depth in a range of 0.015-0.08 mm and a pitch in a range of 0.5-1.2 mm.

12. The invention according to claim 5, wherein the V-shaped groove portions are tilted towards the distal end of the member with a tilt angle in a range of 3°-40°.

13. The invention according to claim 5, wherein the V-shaped groove portions include an area defining a shoulder surface having a radius in a range of 0.02-0.06 mm.

14. The invention according to claim 1, further comprising a depth marker, wherein the depth marker comprises at least one groove portion formed on the outer surface of the member, wherein the depth marker is spaced proximally apart from the first plurality of parallel V-shaped groove portions.

15. An echogenic needle, comprising:
an elongated, cylindrical member having a proximal end and a distal end and an area defining a throughbore formed between the proximal end and the distal end;
wherein the distal end includes an angled portion operable to penetrate human tissue;
a first plurality of helical V-shaped groove portions formed on an outer surface of the member;
wherein the V-shaped groove portions include an angle in a range of 60°-150°;
wherein a bottom surface of the V-shaped groove portions is substantially flat or has a radius equal to or less than 0.05 mm.

16. The invention according to claim 15, wherein the V-shaped groove portions include an angle in a range of 91°-136°.

17. The invention according to claim 15, wherein the first plurality of helical V-shaped groove portions is formed in a first domain in a range of 8-12 mm long.

18. The invention according to claim 17, the first domain of the first plurality of helical V-shaped groove portions commences at 5 mm or less from the distal end of the member.

19. The invention according to claim 18, further comprising a second plurality of helical V-shaped groove portions formed on the outer surface of the member.

20. The invention according to claim 19, wherein the V-shaped groove portions include an angle in a range of 60°-150°.

21. The invention according to claim 20, wherein a bottom surface of the V-shaped groove portions is substantially flat or has a radius equal to or less than 0.05 mm.

22. The invention according to claim 20, wherein the V-shaped groove portions include an angle in a range of 91°-136°.

23. The invention according to claim 19, wherein the second plurality of helical V-shaped groove portions is formed in a second domain in a range of 8-12 mm long.

24. The invention according to claim 23, wherein the first domain is spaced 4-6 mm from the second domain.

25. The invention according to claim 19, wherein the V-shaped groove portions have a depth in a range of 0.015-0.08 mm and a pitch in a range of 0.5-1.2 mm.

26. The invention according to claim 19, wherein the V-shaped groove portions are tilted towards the distal end of the member with a tilt angle in a range of 3°-40°.

27. The invention according to claim 19, wherein the V-shaped groove portions include an area defining a shoulder surface having a radius in a range of 0.02-0.06 mm.

28. The invention according to claim 15, further comprising a depth marker, wherein the depth marker comprises at least one groove portion formed on the outer surface of the member, wherein the depth marker is spaced proximally apart from the first plurality of helical V-shaped groove portions.
